(19) United States
(12) Patent Application Publication
(10) Pub. No.: US 2025/0121156 A1
(43) Pub. Date: Apr. 17, 2025

(54) COMPOSITE MEDICAL BALLOON WITH ENHANCED OUTER FILM LAYER AND RELATED METHODS

(71) Applicant: BARD PERIPHERAL VASCULAR, INC., Franklin Lakes, NJ (US)

(72) Inventor: Corey Stapleton, Gilbert, AZ (US)

(21) Appl. No.: 18/682,041

(22) PCT Filed: Aug. 9, 2022
(86) PCT No.: PCT/US2022/039820

§ 371 Date: Feb. 7, 2024

Related U.S. Application Data
(60) Provisional application No. 63/230,988, filed on Aug. 9, 2021.

(51) Int. Cl. A61M 25/00; A61M 25/10

(52) U.S. Cl. CPC A61M 25/00; A61M 2025/1031; A61M 2025/1075

(57) ABSTRACT

A composite medical balloon (10) includes a base balloon (100) and an outer layer (16) overlying the base balloon, the outer layer comprising a film having a single longitudinal seam (80), or a longitudinal seam facing the base balloon, such as a result of a tube including the seam being everted prior to application to the base balloon. Related methods are also disclosed.

This patent application claims the benefit of U.S. Patent Application No. 63/230,988 filed on Aug. 9, 2021, the disclosure of which is incorporated herein by reference.

### BACKGROUND

Medical balloons are widely used in medical procedures. Typically, an uninflated medical balloon is inserted into a body space. When the medical balloon is inflated, the volume of the medical balloon expands, and the body space is similarly expanded.

In procedures such as angioplasty, the medical balloon may be used to open a collapsed or blocked artery. A typical medical balloon includes a central barrel portion between tapered or conical portions. The medical balloon may be provided in a non-compliant form by including one or more fiber layers, but compliant and semi-compliant forms are also known in the art.

To provide an outer layer for a medical balloon, past proposals have been made. One such proposal involves circumferentially wrapping a thin strip of film in a partially overlapping manner around a base balloon including the cylindrical barrel portion. However, this leads to the creation of a large number of seams along the balloon working surface, which may be undesirable for some applications. The manufacturing process involving the wrapping of a film strip along the entire length of the balloon can be costly, typically requiring an entirely manual process.

Accordingly, a need is identified for a medical balloon that overcomes any or all of the foregoing limitations and possibly others that have yet to be identified.

### SUMMARY

An object of the disclosure is to provide a composite medical balloon including a film outer layer and, in particular, an outer layer (not necessarily the outermost layer) formed of a single piece of film. With still further particularity, the disclosure pertains to a balloon outer film layer comprises a single longitudinal seam.

According to a first aspect of the disclosure, a composite medical balloon comprises a base balloon and an outer layer overlying the base balloon. The outer layer comprises a film having a single longitudinal seam.

In one embodiment, the composite medical balloon includes a fiber layer between the base balloon and the outer layer. The base balloon may comprise first and second tapered portions and a barrel portion therebetween. The film forming the outer layer may comprise a balloon shaped cut-out having a first portion having a first width approximately the same as a diameter of the barrel portion of the base balloon.

In one version, the balloon shaped cut-out comprises a second portion having a second width approximately the same as a diameter of the first tapered portion of the base balloon. The balloon shaped cut-out may further comprise a third portion having a second width approximately the same as a diameter of the second tapered portion of the base balloon.

The single longitudinal seam may comprise overlapping portions of the film bonded together. The outer layer may comprise an everted tube such that the single longitudinal seam faces an outer surface of the base balloon. The outer layer may have a length less than a length of the base balloon.

In one embodiment, the film covers a barrel portion of the base balloon, and further including a different material covering one or both tapered end portions of the balloon. The different material comprises a spirally wrapped film. A catheter may include the composite medical balloon as described herein in any form.

Still another aspect of the disclosure relates to a composite medical balloon, comprising a base balloon and an outer layer overlying the base balloon, the outer layer comprising a film having a longitudinal seam including a bond facing the base balloon. The outer layer may comprise an everted tube.

A further aspect of the disclosure pertains to a method of forming a composite medical balloon. The method comprises providing a base balloon having a barrel portion between first and second tapered portions. The method further comprises forming an outer layer overlying the base balloon, the outer layer comprising a film having a single longitudinal seam.

In one embodiment, the method further includes the step of cutting the outer layer from the film and wrapping the outer layer around the balloon. The method may further include bonding longitudinal edges of the outer layer to form the single longitudinal seam. The forming step may comprise forming a tube having the single longitudinal seam and applying the tube to the balloon. The method may comprise everting the tube.

Still a further aspect of the disclosure pertains to a material for covering a base balloon including tapered end portions and a barrel portion having a first diameter and intermediate the tapered end portions, which have a variable second diameter. The material comprises a single piece of film having end portions with variable widths corresponding to the variable second diameter and an intermediate portion having a width corresponding to the first diameter. The single piece of film has a two-dimensional outline corresponding to but greater in size than a two-dimensional outline of a longitudinal cross-section of the base balloon. A tube may be formed of the material, the tube having a single longitudinal seam. A balloon having an outer layer formed of the material or the tube may also be provided.

Yet a further aspect of the disclosure pertains to a method of forming a composite medical balloon, comprising providing a base balloon having a barrel portion between first and second tapered portions, and forming an outer layer overlying the base balloon, the outer layer comprising a film having a longitudinal seam including a bond facing the base balloon. The method may further comprise the steps of forming a tube of the film including the longitudinal seam; everting the tube; and using the everted tube to form the outer layer overlying the base balloon.

### BRIEF DESCRIPTION OF THE DRAWINGS

The above and further advantages of the disclosure may be better understood by referring to the following description in conjunction with the accompanying drawings in which:

FIG. 1 illustrates a semi-cross section of a medical balloon;

FIG. 2 illustrates an inflated base balloon;

FIG. 3 illustrates a balloon-shaped mandrel;

FIG. 4 illustrates a base balloon having an adhesive layer;

FIG. 5 illustrates a first fiber layer;

FIG. 6 illustrates a cross-section of a base balloon, adhesive layer, and first fiber layer;

FIG. 7 illustrates a cross-section of a base balloon, adhesive layer, and fiber layers;

FIG. 8 illustrates a cross-section of a base balloon, an adhesive layer, a first fiber layer, a second fiber layer, an outer coating layer and a final layer;

FIG. 9 illustrates a fiber-reinforced medical balloon with a longitudinal first fiber layer and a circumferential second fiber layer;

FIG. 10 illustrates a fiber-reinforced medical balloon with a first angled fiber layer and a second longitudinal second fiber layer;

FIG. 11 illustrates a fiber-reinforced medical balloon having a first longitudinal fiber layer and a second angled fiber layer;

FIG. 12 illustrates a fiber-reinforced medical balloon having a longitudinal first fiber layer and an angled second fiber layer;

FIG. 13 illustrates an alternative embodiment of a fiber-reinforced medical balloon;

FIGS. 14, 15, 16, 17, 18, 19, 20, 21, 22, 23, 23A, 24, 25, 26, 26A and 27 illustrate different manners of providing a medical balloon with an outer film layer having a single longitudinal seam partially or entirely covering a base balloon; and

FIGS. 28, 29, 29A, and 30 illustrate a medical balloon with an outer film layer having a single longitudinal seam forming part of a catheter.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of systems and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

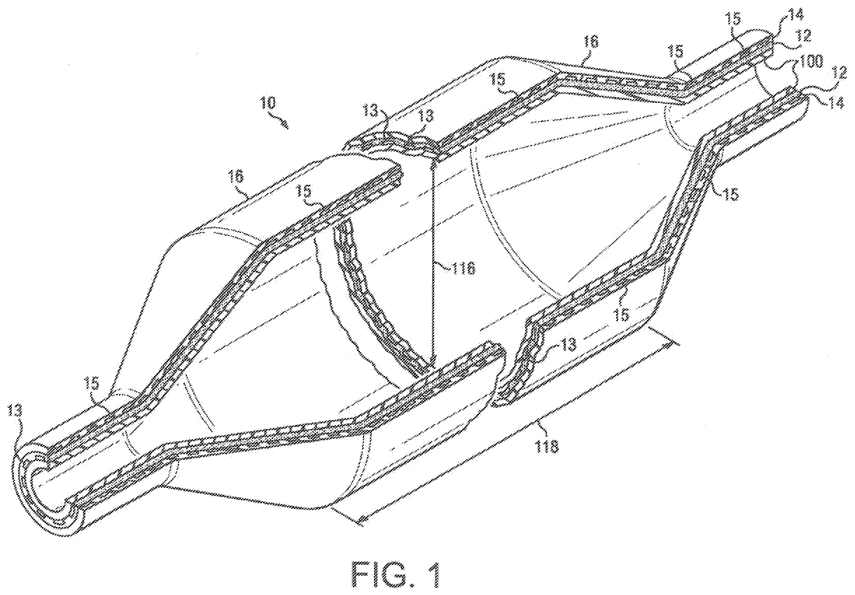

With reference to FIG. 1, a partial cross section of an inflated composite medical balloon 10 is shown. In the illustrated embodiment, and as discussed further in the following description, the balloon 10 may optionally be fiber-reinforced and, as a result, is substantially non-compliant, having limited expansion characteristics. Because the medical balloon 10 is non-compliant, once fully inflated, a diameter 116 of it does not substantially change as the interior pressure increases. As noted further below, the use of a fiber layer is considered entirely optional.

The diameter 116 of an inflated fiber-reinforced medical balloon 10 in accordance with the one embodiment may be about ten millimeters, but may vary depending on the application. The length of an inflated fiber-reinforced medical balloon 10 in accordance with one embodiment may be about eight centimeters. A balloon 10 with a length 118 of 2-200 centimeters or more is possible.

The fiber-reinforced medical balloon 10 may include a base balloon 100 formed of a thin polymer material and a first layer 12 of thin inelastic fibers 13. The balloon 10 may include a second layer 14 made up of one or more fibers 15. An outer layer 16 over the fiber layer(s) 12, 14 may be included, as outlined in the following description.

Each fiber 13 is typically fixed relative to other fibers in the first fiber layer 12 and other fibers in the balloon 10. The thin inelastic fibers 13 of the first fiber layer 12 may be characterized by a high tensile strength, which provide superior burst strength. The fiber-reinforced balloon 10 may also resist abrasion, cuts, and punctures. It may be recognized that enhanced structural integrity may result from the fiber reinforcement.

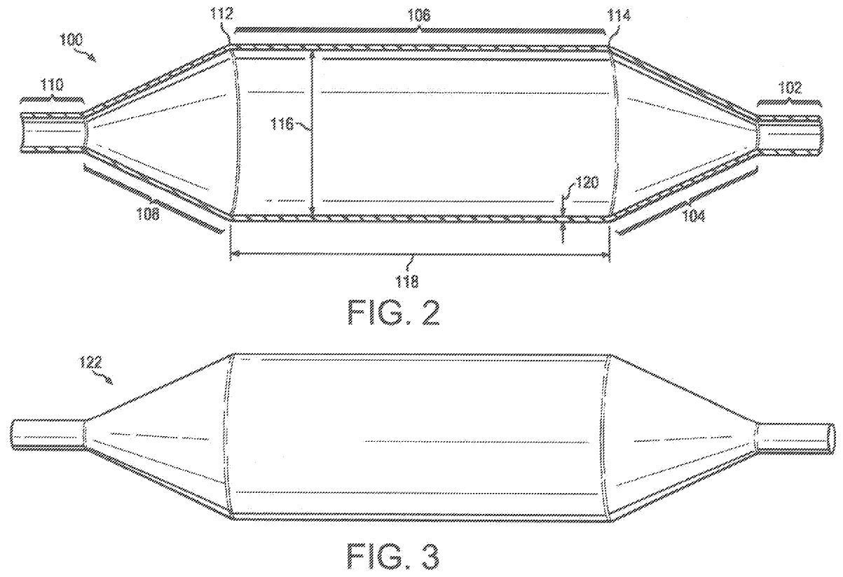

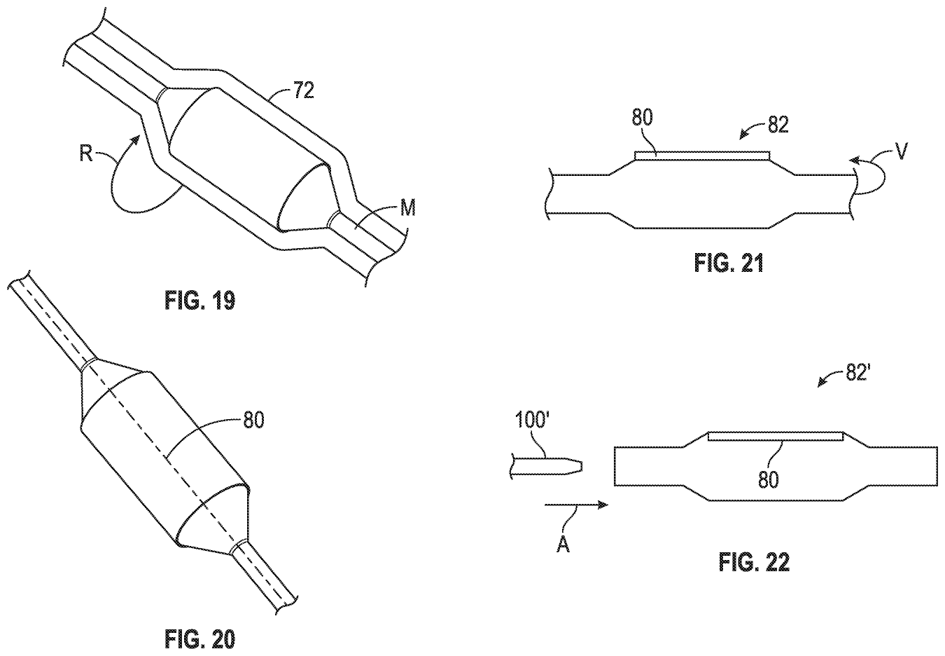

With further reference to FIG. 2, the base balloon 100 may be in the shape of a standard medical balloon, or any other suitable shape. The base balloon 100 typically includes a first neck portion 102 that may be formed as a narrow cylinder fashioned to attach to a catheter shaft (see FIGS. 19, 20 and 21). A second neck portion 110 may be similarly formed as a narrow tube. The first neck portion 102 is formed adjacent to a first cone or tapered portion 104. The first cone portion 104 expands to meet a barrel portion 106 having a working length 118, marked by a first transition 114. The first cone portion 104 is typically constructed at an angle of about twelve to twenty degrees.

The central region or barrel portion 106 meets a second cone or tapered portion 108 at a second transition 112. The second cone portion 108 meets the second neck portion 110. The inclination angle of the tapered or cone portion 108 of an inflated fiber-reinforced medical balloon 10 in accordance with the disclosed embodiment may be about twenty degrees. It will be recognized by those having skill in the art that the fiber-reinforced balloon 10 could be made in a wide variety of diameters 116 and lengths and with a variety of inclinations at the tapered or cone portion 108 of the balloon 10, without limitation.

The base balloon 100 is typically formed of a thin film polymeric material, or other suitable materials with high strength relative to film thickness. Polymers and copolymers that can be used for the base balloon 100 include the conventional polymers and copolymers used in medical balloon construction, such as, but not limited to, polyethylene, (PET), polycaprolactam, polyesters, polyethers, polyamides, polyurethanes, polyimides, ABS, nylons, copolymers, polyester/polyether block copolymers, ionomer resins, liquid crystal polymers, and rigid rod polymers. The base balloon 100 may typically be formed as a blow-molded balloon of a polymer material, such as for example a polyamide, such as nylon.

The base balloon 100 may comprise a polymer, which has been cured into the shape of a balloon, may be formed. This base balloon 100 of a cured polymer may form the inner polymeric wall of the fiber reinforced balloon. With reference to FIG. 3, a removable mandrel 122 may be used as a base for application of the polymer coating. After the polymer is cured, the mandrel 122 may be removed, such as by physical withdrawal, heat, or other known forms of dissolution.

A removable balloon similar to base balloon 100 may be used as the mandrel 122. The mandrel 122 may be made from a variety of materials. The mandrel 122 may be made in the shape of the interior wall of the desired finished balloon. The mandrel 122 may be made of collapsible metal or polymeric bladder, foams, waxes, low-melting metal alloys, and the like. Once the composite balloon is developed and laminated, the mandrel 122 (i.e., base balloon 100) may be removed by melting, dissolving, fracturing, compressing, pressurizing, or other suitable removal techniques.

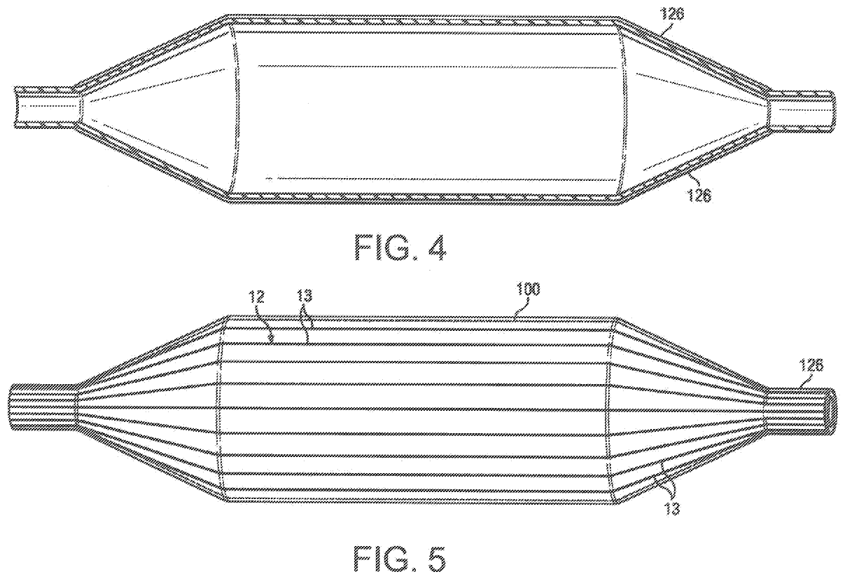

With reference to FIG. 4, it can be understood that a thin coating of an adhesive 126 is applied to the inflated base balloon 100 or to the polymer-coated mandrel 122 prior to applying the first layer inelastic fibers 12. The adhesive 126 binds the fibers 13 sufficiently to hold them in position when the fibers 13 are placed on the base balloon 100. In accordance with one embodiment, a very thin coat of adhesive 126 is applied to the base balloon 100, such as for example 1-MP adhesive, which is a known solution of a polyurethane based polymer and methyl ethyl ketone and methylene chloride, but other forms of adhesive could be used.

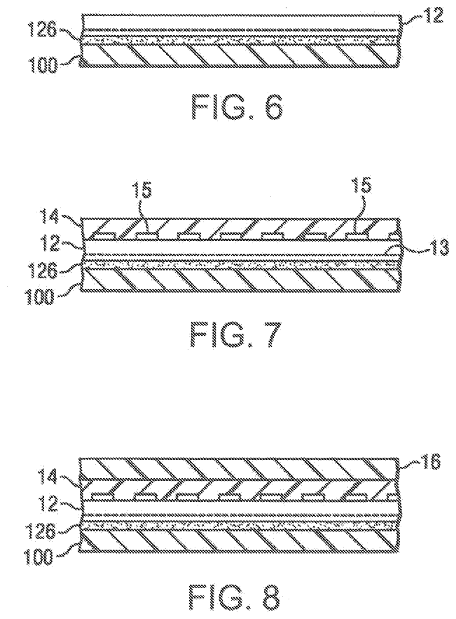

One or more fibers 13 are applied to the base balloon 100 to form a first fiber layer 12, as shown in FIGS. 5 and 6, which may be referred to as the “primary wind.” The fibers 13 of the first fiber layer 12 may be inelastic fiber, typically made of an inelastic fibrous material. An inelastic fiber is a fiber that has very minimal elasticity or stretch over a given range of pressures. Some fibrous materials are generally classified as inelastic although all fibrous material may have a detectable, but minimal, elasticity or stretch at a given pressure. The fibers 13 of the first fiber layer 12 may be high-strength fibers, typically made of a high-strength fibrous material. Some high strength inelastic fibrous materials may include Kevlar, Vectran, Spectra, Dacron, Dyneema, Terlon (PBT), Zylon (PBO), Polyimide (PIM), other ultrahigh molecular weight polyethylene, aramids, and the like.

In a disclosed embodiment, the fibers 13 of the first fiber layer 12 are ribbon-shaped, where the width of the fiber is larger than the thickness of the fiber. The fibers 13 may be flat so that the fiber has a rectangular cross-section. The fibers 13 used in the initial layer of fibers 12 may all be fibers 13 made of the same material and the same shape. Fibers 13 made from different materials may be used in the initial fiber layer 12. Fibers 13 made in different shapes may be used in the initial fiber layer 12. Ultrahigh Molecular Weight (UHMW) Polyethylene fiber 13, which has been flattened on a roll mill may be used to form the first fiber layer 12. To the flattened fiber 13 is applied an adhesive, such as the 1-MP adhesive. The fibers 13 may be arranged as 30 longitudinal fibers, each substantially equal in length to the length of the balloon 10.

The fibers 13 of the initial fiber layer 12 may be arranged so that each fiber 13 is substantially parallel to the long axis of the balloon 10. The density of the fibers 13 in the initial fiber layer 12 is determined by the number of fibers 13 or fiber winds per inch and the thickness of the fibers 13. In a disclosed embodiment of the first fiber layer 12 having longitudinally placed fibers 13, a fiber density of generally about 15 to 30 fibers 13 having a fiber thickness of about 0.0005 to 0.001 inch and placed equidistant from one another provide adequate strength for a standard-sized fiber-reinforced medical balloon 10. Each of the fibers 13 is substantially equal in length to the balloon 10. The first fiber layer 12 may prevent longitudinal extension of the completed fiber-reinforced balloon 10.

In accordance with a disclosed embodiment, a second fiber layer 14 made with one or more high-strength inelastic fibers 15 is positioned along circumference of the balloon 10, as shown in FIG. 7. The circumferentially placed fibers 15 may be transverse or substantially transverse to the longitudinal axis of the balloon 10. The circumferential fibers 15 may prevent or minimize distension of the balloon diameter 116 at pressures between the minimal inflation pressure and the balloon burst pressure.

The fibers 15 of the second fiber layer 14 may be inelastic fiber, typically made of an inelastic fibrous material. An inelastic fiber is a member of a group of fibers that have very minimal elasticity or stretch in a given range of pressures. Some fibrous materials are generally classified as inelastic although all fibrous material may have a detectable, but minimal elasticity or stretch at a given pressure. The fibers 15 of the second fiber layer 14 may be high-strength fibers, typically made of a high-strength fibrous material. Some high strength inelastic fibrous materials may include Kevlar, Vectran, Spectra, Dacron, Dyneema, Terlon (PBT), Zylon (PBO), Polyimide (PIM), other ultra-high molecular weight polyethylene, aramids, and the like.

In a disclosed embodiment, the fibers 15 of the second fiber layer 14 are ribbon-shaped, where the width of the fiber is larger than the thickness of the fiber. The fibers 15 may be flat so that the fiber has a rectangular cross-section. The fibers 15 used in the second layer of fibers 14 may all be fibers 15 made of the same material and the same shape. Fibers 15 made from different materials may be used in the second fiber layer 14. Fibers 15 made in different shapes may be used in the second fiber layer 14. UHMW polyethylene fiber 15, which has been flattened on a roll mill may be used to form the second fiber layer 14. To the flattened fiber 15 is applied a thin coat of an adhesive, such as the 1-MP adhesive. The fibers 15 may be arranged as a second fiber layer 14 may have a fiber density of 54 wraps per inch.

The fibers 15 of the second fiber layer 14 may be perpendicular to or substantially perpendicular to the fibers 13 placed longitudinally to form the first fiber layer 12. This transverse placement of the first fiber layer 12 and the second fiber layer 14 allows for maximum radial stability of the fiber-reinforced balloon 10. The placement of the fiber layers 12 and 14 distributes the force on the balloon surface equally, creating pixelized pressure points of generally equal shape, size, and density.

The fibers 13 of the first fiber layer 12 may be the same as or different from the fiber 15 of the second fiber layer 14. Specifically, the fibers 15 of the second fiber layer 14 may be made of a different material or materials than the fibers 13 of the first layer 12. The fibers 15 of the second layer 14 may be shaped differently from the fibers 13 of the first fiber layer 12. The characteristics of the fibers or combination of fibers used for the first or second fiber layers may be determined from the specific properties required from the resulting fiber-reinforced balloon 10.

With respect to the fiber density of the second fiber layer 14, in accordance with the disclosed embodiment, fiber 15 having a thickness of about 0.0005 to 0.001 inch and arranged in parallel lines with about 50 to 80 wraps per inch provides generally adequate strength. A single fiber 15 may form the second fiber layer 14, with the fiber 15 wound in a generally parallel series of circumferential continuous loops.

With reference to FIG. 8, a cross section of the integral layers of a fiber-reinforced medical balloon 10 is shown. The first fiber layer 12 and the second fiber layer 14 may be coated with an outer layer 16. The outer layer 16 may be, in the disclosed embodiment, a hybrid outer layer, as outlined further in the following description. A composite structure typically including a base balloon 100, an adhesive 126, a first fiber layer 12, a second fiber layer 14 and an outer layer 16 forming a composite, non-compliant fiber-reinforced balloon 10 particularly suitable for medical uses. Typically, the fibers 13 and 15 are fixed when the fiber-reinforced balloon 10 is initially deflated, and then subsequently inflated and deflated during use.

With reference to FIG. 9, a fiber reinforced balloon 10 in accordance with the disclosed embodiment, is shown. In this embodiment, the fibers 13 are parallel to the balloon 10 long axis.

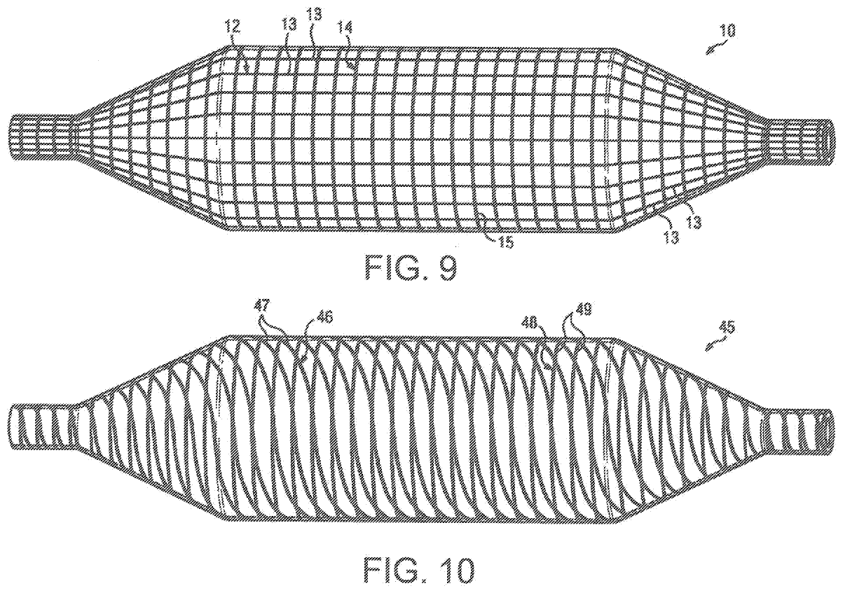

With reference to FIG. 10, a fiber reinforced balloon 45, in accordance with another embodiment is shown. The fiber-reinforced balloon 45 may include a first fiber layer 46 with fibers 47 that lie at an angle to the longitudinal axis of the balloon 45. In this embodiment, neither the fibers 47 of the first fiber layer 46 nor the fibers 49 of the second fiber layer 48 are positioned parallel to the longitudinal axis of the balloon 45. In accordance with one embodiment, the fibers 47 of the first fiber layer 46 may be positioned parallel to a line at a five-degree angle to a line parallel to the longitudinal axis of the balloon 10. In accordance with another embodiment, the fibers 47 of the first fiber layer 46 may be positioned parallel to a line at a twenty-degree angle to a line parallel to the longitudinal axis of the balloon 10.

In accordance with another embodiment, the fibers 47 of the first fiber layer 46 may be positioned parallel to a line at a thirty-degree angle to a line parallel to the longitudinal axis of the balloon 10. In accordance with another embodiment, the fibers 47 of the first fiber layer 46 may be positioned parallel to a line at a forty-five-degree angle to a line parallel to the longitudinal axis of the balloon 10. It will be apparent to those having skill in the art that the fibers 47 may be placed at any appropriate angle.

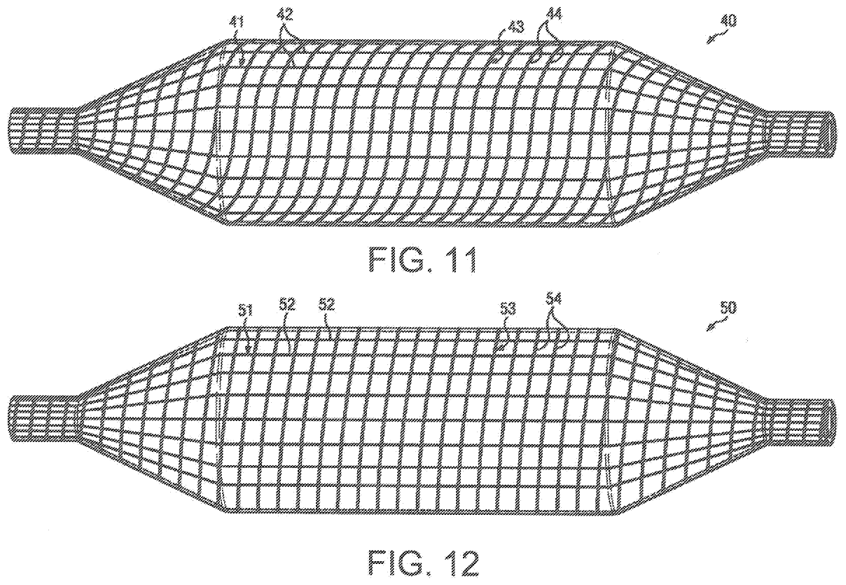

The fibers 49 of the second fiber layer 48 lie parallel to the circumference of the balloon 10. With reference to FIG. 11, a fiber-reinforced balloon 40 in accordance with another embodiment is shown. The fiber reinforced balloon 40 may include a second fiber layer 43 with fibers 44 that lie at an angle to the circumference of the balloon 40. In accordance with one embodiment, the fibers 44 of the second fiber layer 43 may be positioned parallel to a line at a five-degree angle to a line parallel to the circumference of the balloon 10.

The fiber 44 of the second fiber layer 43 may be positioned parallel to a line at a twenty-degree angle to a line parallel to the circumference of the balloon 10. In accordance with one embodiment, the fiber 44 of the second fiber layer 43 may be positioned parallel to a line at a thirty-degree angle to a line parallel to the circumference of the balloon 10. In accordance with one embodiment, the fiber 44 of the second fiber layer 43 may be positioned parallel to a line at a forty-five-degree angle to a line parallel to the circumference of the balloon 10. It will be apparent to those skilled in the art that the fibers 44 may be placed at any appropriate angle.

The fibers 42 of the first fiber layer 41 and the fibers 44 of the second fiber layer 43 are positioned perpendicularly relative to each other. With reference to FIG. 12, a fiber-reinforced balloon 50 in accordance with another embodiment is shown. A fiber-reinforced balloon 50 may include fibers 52 of the first fiber layer 51 and fibers 54 of the second fiber layer 53 positioned relatively at an angle other than a right angle.

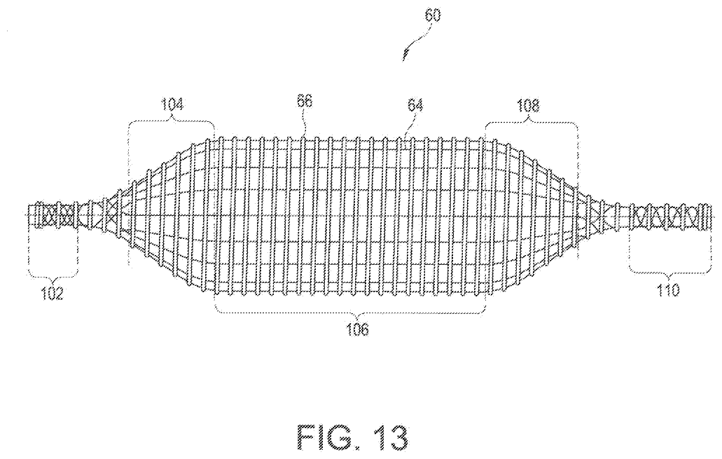

Referring to FIG. 13, a medical balloon 60 may also include a single continuous fiber forming a longitudinal fiber strand 64 and a hoop fiber strand 66. Specifically, the fiber 62 may be wrapped circumferentially around one end portion, such as neck portion 102, and directed longitudinally to the other end portion, or neck portion 110 (possibly at a non-zero angle to the longitudinal axis). The fiber 62 is then wrapped around the other end portion 110, and returned in the opposite longitudinal direction (again, possibly at a non-zero angle). The single continuous fiber 62 can continue to be wound back and forth along the balloon 60 for a desired number of longitudinal passes, and then would circumferentially or helically around the balloon 60 at a desired fiber pitch. A more complete description of the application to a single continuous fiber to a medical balloon 60 may be found in U.S. Pat. No. 10,485,949.

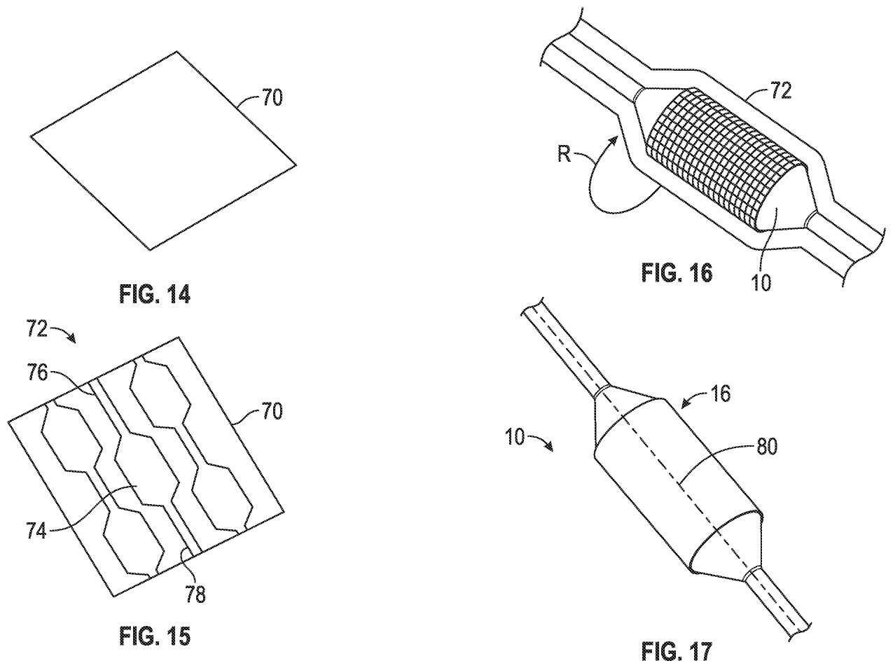

Turning to FIGS. 14-18, a manner of forming the outer layer 16 of the balloon 10 is shown (which may apply to any of the embodiments of balloons described herein). The balloon 10 as illustrated is fiber-reinforced, and thus includes one or more fiber layers, and any mandrel may be removed, if present. As illustrated in FIG. 14, a single piece of film 70 may be provided, which may comprise a flat square shaped piece as shown, but could take any shape. The film 70 may be fabricated of the same material as the base balloon 100 (e.g., a nylon or PET film), or a different material may be used.

A section of the film 70 corresponding to the base balloon 100 in size and shape may then be cut out of the film. Specifically, a two-dimensional balloon-shaped cut-out 72 may be formed having at least a wider portion 74 for fully covering the cylindrical barrel portion 106 of the balloon 10, between narrower portions 76, 78 for covering at least the tapered or conical portions 104, 108 and possibly also the necks. Each portion 74, 76, 78 of the cut-out 72 should have a cross dimension, or width W, at least as great as a diameter D of the portion of the balloon 10 to be covered (and would be progressively variable in order to fully cover the tapered portions 104, 108 of the base balloon 100). As can be appreciated from FIG. 15, multiple cut-outs may be formed from a single sheet of film 70, and may be applied to one or more base balloons to create one or more outer layers thereupon.

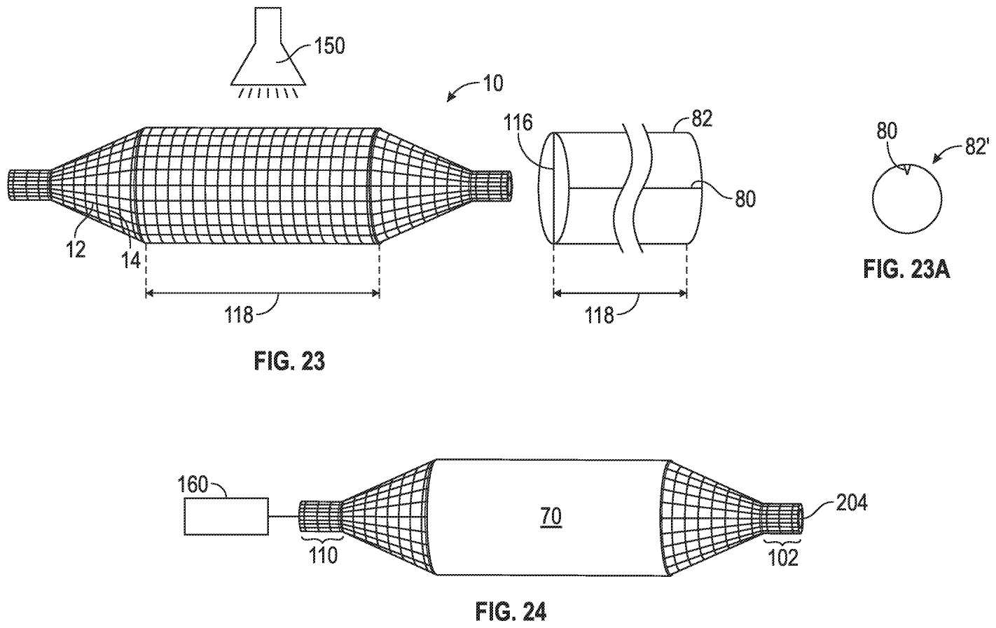

The as-formed balloon 10 is then provided with an adhesive coating, such as 1-MP, and at least partially deflated. The application of the adhesive coating to the base balloon 100 may be done by spraying using a sprayer 150 (see FIG. 23), but could also be done using other forms of application, such as dip coating, sputtering, brushing, rolling, or the like.

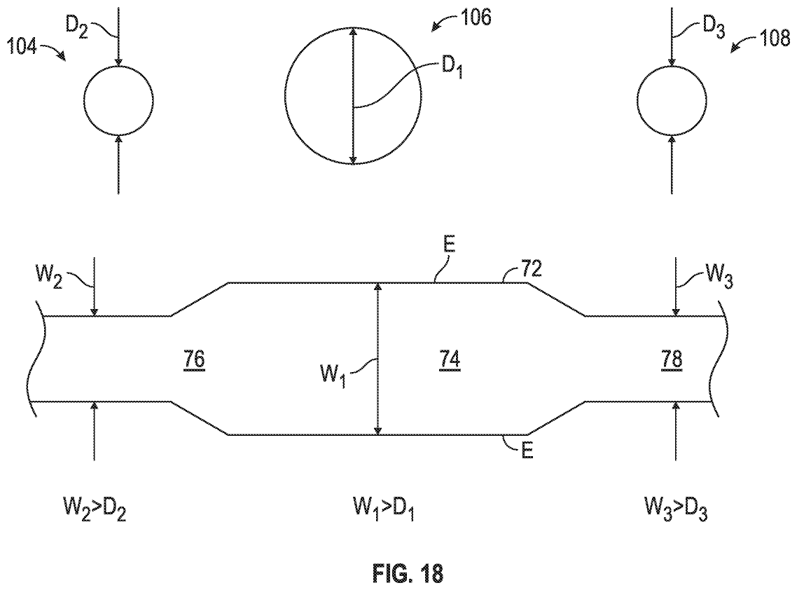

The balloon-shaped cut-out 72 is then wrapped around the base balloon 100 with the applied adhesive to form the outer layer (note arrow R in FIG. 16) with the longitudinally cut edge portions E (see FIG. 18) overlapping. The cut longitudinal edge portions E may then be bonded together, such as by laser welding, and trimmed if desired. In view of the fact that the cut-out 72 is fabricated from a single piece of material having the overall shape of the balloon, a single longitudinal seam 80 results.

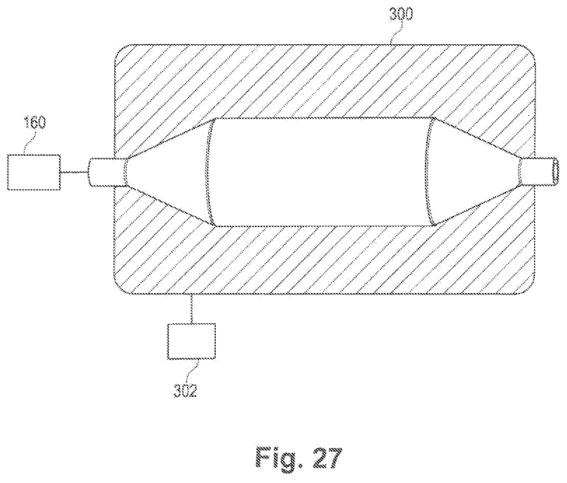

With the outer layer 16 in place, the balloon 10 may then be subjected to lamination or consolidation, such as by placing it within a split die 300, as shown in FIG. 27. Heat and pressure may then be applied (note inflation source 202 and heater 203 connected to die 300) to the balloon 10 to activate the previously applied adhesive and allow it to flow along the interface between the outer layer 16 and the underlying base balloon 100. Consequently, the outer layer 16 becomes fully bonded in place and connected to the underlying layer, such as base balloon 100 or any fiber layers 12, 14 present.

Advantageously, this creates a balloon 10 having an outer layer 16 with several distinct advantages. Specifically, the ability to provide an outer layer 16 with only a single seam allows for the selection of a material with superior properties (e.g., porosity, texture, hardness, etc.) in terms of any associated treatment, such as delivery of a treatment agent or drug (e.g., paclitaxel) coated on the barrel portion, or stent retention/release. This further avoids the need for crimping or folding material around the cone portions 104, 108, which can result in bunching of material when the balloon is laminated and/or folded. This bunched material can also unfurl upon balloon expansion during manufacturing and cause process issues, avoided by using spiral wrapping.

With reference to FIGS. 19-22, an alternative approach is to wrap the cut-out 72 around a mandrel M or other scaffolding having the shape of the balloon 10, with the longitudinally cut ends overlapping. The cut-out 72 may then be welded on the mandrel, which may be removed (dissolved, deflated, etc.). The resulting tube 82 may then be everted (note tube 82′) so that the formed seam is on the inside of the tube (FIG. 23A). The deflated, possibly folded base balloon 100′ may then be inserted into the tube 82′ and inflated, as shown in FIG. 23, with the bond or weld of the formed seam 80 facing the base balloon 100′.

Still another alternative is to form the tube so as to correspond to only a portion of the base balloon 100, such as the barrel portion 106. Specifically, as shown in FIG. 23, the tube 82 may be tubular in shape, and positioned over the adhesive coating applied to the base balloon 100 and any overlying fiber layers 12, 14. As perhaps best shown in FIG. 24, the tube 82 has a length 118 corresponding to only the barrel portion 106 and an inner diameter 116 (FIG. 23) equal to or greater than the outer diameter of the base balloon 100, or may be smaller in diameter and stretched by the base balloon 100 on inflation.

The balloon 10 with the adhesive coating and the tube 82 in place over the barrel portion 106 is then inflated. As indicated in FIG. 24, this may be achieved by supplying a pressurized fluid (air) from a source 160 through one neck portion 110 and providing a seal 204 at the open end of the other neck portion 102.

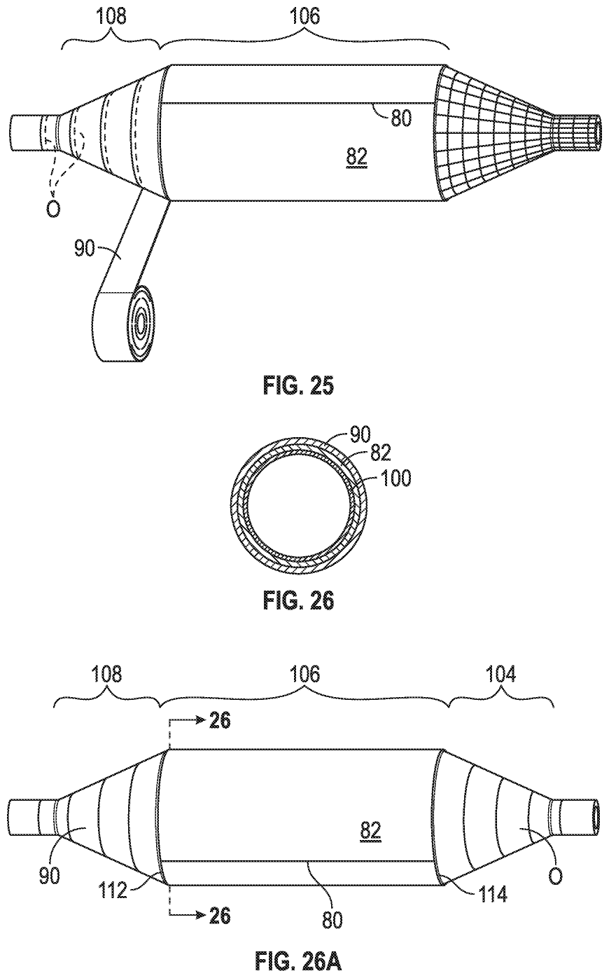

With the balloon 10 inflated, a different material may be applied to at least one of the cone portions 104, 108 in the same layer as the tube 82 in the barrel portion 106. Specifically, as shown in FIGS. 25 and 26, a ribbon of material 90, such as one comprising a polymer film, such as for example pre-stretched PEBAX film. The material 90 may have a thickness similar to that of the tube 82, and may be spirally wrapped or wound around one or both of the cone portions 104, 108, so as to form a spiral wrapping. The width of the material 90 may be relatively narrow, such that two or more passes or winds are required to cover the entirety of each cone portions 104, 108, but the arrangement will ultimately depend on the relative dimensions of the material 90 and the cone portions 104, 108. Adhesive may also be applied to the material 90 during spiral wrapping, but may alternatively or additionally be applied directly to the cone portions 104, 108.

The winding of the material 90 may be done in an overlapping manner. Specifically, each wind or pass of the ribbon of material 90 may at least partially overlap with an adjacent wind (as indicated by phantom lines O indicating overlap). Furthermore, the ribbon of material 90 may overlap with the edges of the tube 82 formed of material 70 at the transitions 112, 114 adjacent to the cone portions 104, 108, as can be understood by the cross-sectional view of FIG. 26A. Alternatively, if the ribbon of material 90 is applied first, then the edges of the tube 82 could overlap with it instead. Adhesive may be added between overlapped regions to aid in seamless bonding and transition between regions.

With the tube 82 any spiral wrapping of material 90 in place, the balloon 10 may then be subjected to lamination or consolidation, such as by placing it within a split die 300, as shown in FIG. 27. Heat and pressure may then be applied (note inflation source 202 and heater 203 connected to die 300) to the balloon 10 to activate the previously applied adhesive and allow it to flow along the interface between the tube 70 and material 80 and the underlying base balloon 100. Consequently, the tube 70 and the spirally wrapped material 80 becomes fully bonded in place and connected to the underlying layer, such as base balloon 100 or any fiber layers 12, 14 present.

Advantageously, this creates a balloon 10 having a hybrid outer layer 16 with several distinct advantages. Specifically, the ability to provide a tube 82 with only a single seam 80 for the barrel portion 106 allows for the selection of a material with superior properties (e.g., porosity, texture, hardness, etc.) in terms of any associated treatment, such as delivery of a treatment agent or drug (e.g., paclitaxel) coated on the barrel portion, or stent retention/release. The use of a different material for covering the cone portions 104, 108 allows for these regions of the composite balloon 10 to be provided with different properties, such as enhanced flexibility or a differential thickness. Using a spiral wrapping further avoids the need for crimping or folding material around the cone portions 104, 108.

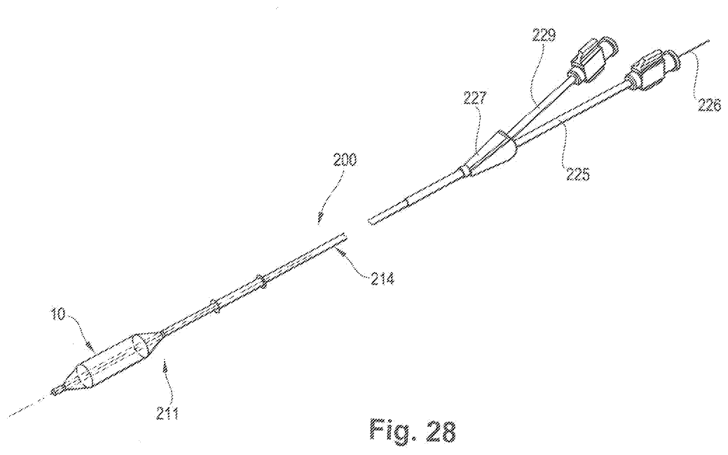

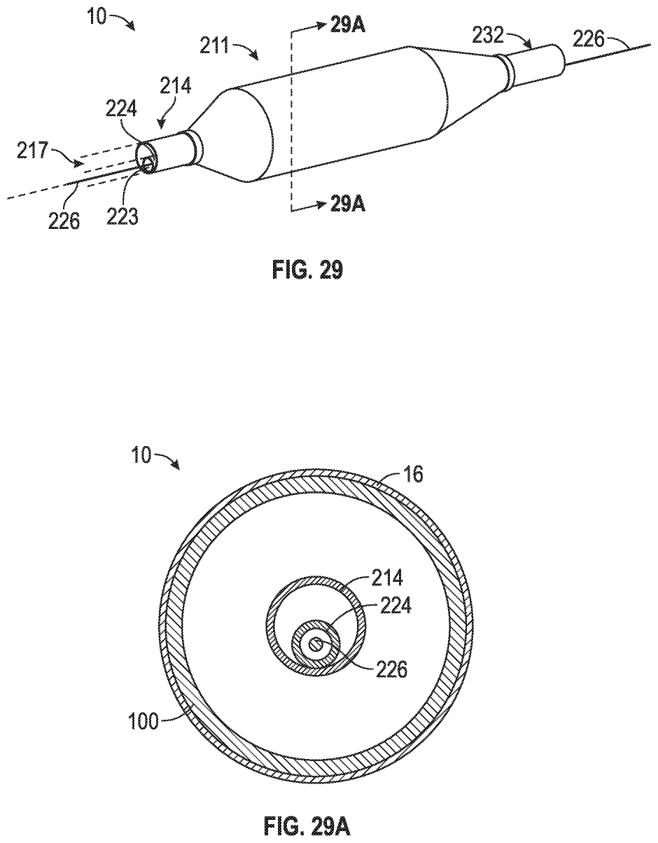

Turning to FIGS. 28, 29, and 30, it can be understood that the balloon 10 as formed using any of the above-described techniques may form part of a catheter 200 having a shaft 214 with a distal end portion 211 to which the balloon 10 is mounted. The balloon 10 is sealed at balloon ends to allow the inflation via one or more inflation lumens 217 extending within catheter shaft 214 and communicating with the interior of the balloon 10. The catheter 200 may also include a guidewire lumen 223 formed by a shaft 224, which may be within the shaft 214 and, more particularly, within the inflation lumen 217. This lumen 223 directs the guidewire 226 through the catheter 200 (see FIG. 29A), and along the distal end portion of which the balloon 10 may be located, including through a tip 232 distal of balloon 10 distal end.

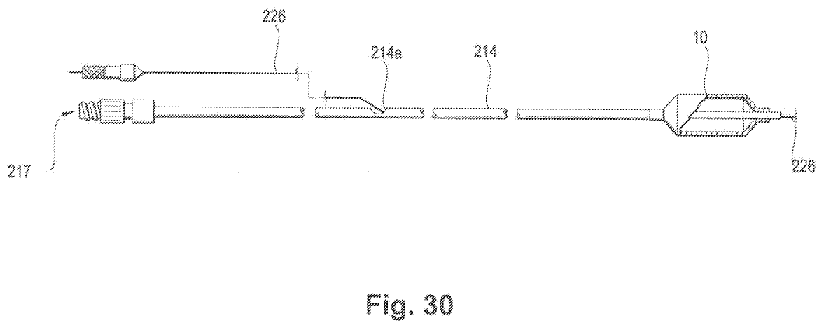

As illustrated in FIG. 29, this guidewire 226 may extend through the proximal end portion of the catheter 200 via a first port 225 of a connector or hub 227 at a proximal end portion 213 of the shaft 214 into the lumen 223 to achieve an “over the wire” (OTW) arrangement, but could also be provided in a “rapid exchange” (RX) configuration (in which the guidewire 226 exits from the shaft 214 at an optional lateral opening 214a (see in FIG. 30) closer to the distal end but proximal of balloon 10) or else is fed through the tip 232 at a passage distally of the balloon 10 (“short” RX; not shown). A second port 229 may also be associated with catheter 200, such as by way of connector 227, for introducing a fluid (e.g., saline, a contrast agent, or both) into the interior compartment of the balloon 10 via the inflation lumen 217.

While the embodiments above and shown and described as a balloon having a fiber layer, it should be understood that this is considered an optional feature. Thus, the balloon 10 could simply comprise the base balloon 100 and the outer layer 16, as proposed, without the inclusion of fibers. Furthermore, any type of intermediate layer may be provided between the base balloon 100 and the outer layer 16 to provide the resulting balloon with desired characteristics, such as a particular degree of compliance or an enhanced resistance to bursting.

Summarizing, this disclosure may be considered to relate to the following items:

1. A composite medical balloon, comprising:
a base balloon; and
an outer layer overlying the base balloon, the outer layer comprising a film having a single longitudinal seam.
   2. The composite medical balloon of item 1, further including a fiber layer between the base balloon and the outer layer.
   3. The composite medical balloon of item 1 or item 2, wherein the base balloon comprises including first and second tapered portions and a barrel portion therebetween.
   4. The composite medical balloon of any of items 1-3, wherein the film forming the outer layer comprises a balloon shaped cut-out having a first portion having a first width approximately the same as a diameter of the barrel portion of the base balloon.
   5. The composite medical balloon of any of items 1-4, wherein the balloon shaped cut-out comprises a second portion having a second width approximately the same as a diameter of the first tapered portion of the base balloon.
   6. The composite medical balloon of any of items 1-5, wherein the balloon shaped cut-out comprises a third portion having a second width approximately the same as a diameter of the second tapered portion of the base balloon.
   7. The composite medical balloon of any of items 1-6, wherein the single longitudinal seam comprises overlapping portions of the film bonded together.
   8. The composite medical balloon of any of items 1-7, wherein the outer layer comprises an everted tube such that the single longitudinal seam faces an outer surface of the base balloon.
   9. The composite medical balloon of any of items 1-8, wherein the outer layer has a length less than a length of the base balloon.
   10. The composite medical balloon of any of items 1-9, wherein the film covers a barrel portion of the base balloon, and further including a different material covering one or both tapered end portions of the balloon.
   11. The composite medical balloon of item 10, wherein the different material comprises a spirally wrapped film.
   12. A catheter including the composite medical balloon of any of items 1-11.
   13. A composite medical balloon, comprising:
a base balloon; and
an outer layer overlying the base balloon, the outer layer comprising a film having a longitudinal seam formed by a bond facing the base balloon.
   14. The composite medical balloon of item 13, wherein the outer layer comprises an everted tube.
   15. A method of forming a composite medical balloon, comprising:
providing a balloon having a barrel portion between first and second tapered portions;
forming an outer layer overlying the balloon, the outer layer comprising a film having a single longitudinal seam.
   16. The method of item 15, further including the step of cutting the outer layer from the film and wrapping the outer layer around the balloon.
   17. The method of item 15 or item 16, further including the step of bonding longitudinal edges of the outer layer to form the single longitudinal seam.
   18. The method of any of items 15-16, wherein the forming step comprises forming a tube having the single longitudinal seam and applying the tube to the balloon.
   19 The method of any of items 15-18, further including the step of everting the tube.
   20. A method of forming a composite medical balloon, comprising:
providing a base balloon having a barrel portion between first and second tapered portions; and
forming an outer layer overlying the base balloon, the outer layer comprising a film having a longitudinal seam facing the base balloon.
   21. The method of item 20, further comprising the step of:
forming a tube of the film including the longitudinal seam;
everting the tube;
using the everted tube to form the outer layer overlying the base balloon.
   22. A material for covering a base balloon including tapered end portions and a barrel portion having a first diameter and intermediate the tapered end portions, which have a variable second diameter, comprising:

a single piece of film having end portions with variable widths corresponding to the variable second diameter and an intermediate portion having a width corresponding to the first diameter.

23. The material of item 22, wherein the single piece of film has a two-dimensional outline corresponding to but greater in size than a two-dimensional outline of a longitudinal cross-section of the base balloon.
24. A tube forming of the material of item 22, having a single longitudinal seam.
25. A balloon including an outer layer formed of the material of item 22 or the tube of item 24.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g., component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. For example, the term “outer” does not necessarily mean outermost, and while the disclosed film layer may be the outermost layer, it could also be an inner layer, or multiple of such layers may be provided. All publications, patents and patent applications mentioned in this specification are herein incorporated in their entirety by reference into the specification, to the same extent as if each individual publication, patent, or patent application was specifically and individually indicated to be incorporated herein by reference. In addition, or identification of any reference in this application shall not be construed as an admission that such reference is available as prior art to the present disclosure.

1. A composite medical balloon, comprising:
a base balloon comprising first and second tapered portions and a barrel portion there between, a first neck portion adjacent the first tapered portion, and a second neck portion adjacent the second tapered portion; and
an outer layer overlying the base balloon, the outer layer comprising a film including a wider portion for fully covering the barrel portion, and narrower portions for covering the first and second tapered portions, the first neck portion and the second neck portion, the film having a single longitudinal seam.

2. The composite medical balloon of claim 1, further including a fiber layer between the base balloon and the outer layer.

3. The composite medical balloon of claim 1, wherein the first neck portion is formed as a narrow cylinder fashioned to attach to a catheter shaft.

4. The composite medical balloon of claim 3, wherein the film forming the outer layer comprises a balloon shaped cut-out having a first portion having a first width approximately the same as a diameter of the barrel portion of the base balloon.

5. The composite medical balloon of claim 4, wherein the balloon shaped cut-out comprises a second portion having a second width approximately the same as a diameter of the first tapered portion of the base balloon.

6. The composite medical balloon of claim 4, wherein the balloon shaped cut-out comprises a third portion having a second width approximately the same as a diameter of the second tapered portion of the base balloon.

7. The composite medical balloon of claim 1, wherein the single longitudinal seam comprises overlapping longitudinal edge portions of the film bonded together.

8. The composite medical balloon of claim 1, wherein the outer layer comprises an everted tube such that the single longitudinal seam faces an outer surface of the base balloon.

9. The composite medical balloon of claim 1, wherein the outer layer has a length less than a length of the base balloon.

10. The composite medical balloon of claim 1, wherein the film covers a barrel portion of the base balloon, and further including a different material covering one or both tapered end portions of the balloon.

11. The composite medical balloon of claim 10, wherein the different material comprises a spirally wrapped film.

12. A catheter including the composite medical balloon of claim 1.

13. A composite medical balloon, comprising:
a base balloon comprising a first neck portion adjacent a first tapered portion, the first tapered portion meeting a barrel portion, a second tapered portion, the second tapered portion meeting a second neck portion; and
an outer layer overlying the base balloon, the outer layer comprising a film including a wider portion for fully covering the barrel portion, narrower portions for covering the first and second tapered portions, the first neck portion and the second neck portion, the film having a longitudinal seam formed by a bond facing the base balloon.

14. The composite medical balloon of claim 13, wherein the outer layer (Original) comprises an everted tube.

15. A method of forming a composite medical balloon, comprising:
providing a base balloon having a barrel portion between first and second tapered portions, a first neck portion adjacent the first tapered portion, and a second neck portion adjacent the second tapered portion; and
forming an outer layer overlying the base balloon, the outer layer comprising a film including a wider portion for fully covering the barrel portion, narrower portions for covering the first and second tapered portions, the first neck portion and the second neck portion, the film having a single longitudinal seam.

16. The method of claim 15, further including the step of cutting the outer layer from the film and wrapping the outer layer around the base balloon.

17. The method of claim 15, further including the step of bonding longitudinal edges of the outer layer to form the single longitudinal seam.

18. The method of claim 15, wherein the forming step comprises forming a tube having the single longitudinal seam and applying the tube to the base balloon.

19. The method of claim 18, further including the steps of everting the tube and then applying the everted tube to the base balloon.

20. A method of forming a composite medical balloon, comprising:
providing a base balloon having a barrel portion between first and second tapered portions, a first neck portion adjacent the first tapered portion, and a second neck portion adjacent the second tapered portion; and
forming an outer layer overlying the base balloon, the outer layer comprising a film including a wider portion for fully covering the barrel portion, narrower portions for covering the first and second tapered portions, the first neck portion and the second neck portion, the film having a longitudinal seam with a bond facing the base balloon.

21. The method of claim 20, further comprising the steps of:
forming a tube of the film including the longitudinal seam;
everting the tube; and
using the everted tube to form the outer layer overlying the base balloon.

22-23. (canceled)

24. A tube formed of the material of claim 18, having a single longitudinal seam.

25. (canceled)
